(19) United States
(12) Patent Application Publication
(10) Pub. No.: US 2018/0104101 A1
(43) Pub. Date: Apr. 19, 2018

(54) FORMED CUTTER FOR VITRECTOMY PROBE

(71) Applicant: Novartis AG, Basel (CH)

(72) Inventor: Jose Luis Lopez, Cypress, CA (US)

(21) Appl. No.: 15/784,278

(22) Filed: Oct. 16, 2017

Related U.S. Application Data
(60) Provisional application No. 62/410,008, filed on Oct. 19, 2016.

(51) Int. Cl. A61F 9/007

(52) U.S. Cl. CPC A61F 9/00763

(57) ABSTRACT

Vitreoretinal probes and methods related thereto are disclosed herein. A vitreoretinal probe may include a needle and a cutter slidably disposed within the needle. The cutter may include a tubular body having a distal end. The distal end may include a first section having an outer diameter that is greater than an outer diameter of the tubular body and a second section having an outer diameter that is not in contact with an inner surface of the needle.

This application claims the benefit of priority of U.S. Provisional Patent Application Ser. No. 62/410,008 titled “Formed Cutter for Vitrectomy Probe”, filed on Oct. 19, 2016, whose inventor is Jose Luis Lopez, which is hereby incorporated by reference in its entirety as though fully and completely set forth herein.

### BACKGROUND

Microsurgical procedures may frequently require precision cutting and/or removing various body tissues. For example, certain ophthalmic surgical procedures may require cutting and removing portions of the vitreous humor, a transparent jelly-like material that fills the posterior segment of the eye. The vitreous humor, or vitreous, is composed of numerous microscopic fibrils that are often attached to the retina. Therefore, cutting and removing the vitreous must be done with great care to avoid traction on the retina, the separation of the retina from the choroid, a retinal tear, or, in the worst case, cutting and removal of the retina itself. In particular, delicate operations such as mobile tissue management (e.g., cutting and removal of vitreous near a detached portion of the retina or a retinal tear), vitreous base dissection, and cutting and removal of membranes may be particularly difficult.

Vitrectomy probes may typically be inserted via an incision in the sclera near the pars plana. The surgeon may also insert other microsurgical instruments, such as a fiber optic illuminator, an infusion cannula, or an aspiration probe during the posterior segment surgery. While performing the surgery, the surgeon may view the eye under a microscope. Vitrectomy probes may typically include a needle and a cutter arranged coaxially with and movably disposed within the needle, and a port extending radially through the needle near the distal end thereof. Vitreous and/or membranes may be aspirated into the open port, and the cutter may be actuated, closing the port. Upon the closing of the port, cutting surfaces on both the needle and the cutter may cooperate to cut the vitreous and/or membranes, and the cut tissue may then be aspirated away through the cutter.

Typically, vitrectomy probes may have a flare at the cutting end in order for the shearing edges of the cutter and needle to make full contact with each other and cut properly. If there is no contact at any point, then cutting may be compromised. Although, the flare may be beneficial for cutting in single blade cutters (no port in the cutter), it may be detrimental for dual blade cutters by creating a gap during its return stroke.

### SUMMARY

In an exemplary aspect, the present disclosure is directed to a vitrectomy probe including a needle and a cutter slidably disposed within the needle. The cutter may include a tubular body having a distal end. The distal end may include a first section having an outer diameter that is greater than an outer diameter of the tubular body and a second section having an outer surface that is not in contact with an inner surface of the needle. The cutter may include a side cutout in the distal end. The side cutout may be configured to reduce friction between the needle and the cutter. The side cutout may be formed in a lower portion of the distal end. The cutter may include a lateral port in the tubular body. The cutter may include a distal port in the distal end of the tubular body. The cutter may also include a proximal cutting edge and a distal cutting edge. The proximal cutting edge may be formed by a distal side of the lateral port, and the distal cutting edge may be formed by a distal side of the distal end. The second section may include two or more flat sections on an outer surface of the distal end. The cutter may include a bend ranging from about 2° to about 5°. The bend may be configured to allow a contact portion between an inner surface of the needle and the first section of the cutter. The vitrectomy probe may also include a housing that may be attached to proximal ends of the cutter and the needle. The vitrectomy probe may also include an interior channel in the cutter to aspirate dissected tissue.

In another exemplary aspect, the present disclosure is directed to a method for operating a vitrectomy probe. A method for operating a vitrectomy probe may include positioning the vitrectomy probe in an eye. The vitrectomy probe may include a needle and a cutter slidably disposed within the needle. The cutter may include a tubular body and a distal end. The distal end may include a first section having an outer diameter that is greater than an outer diameter of the tubular body and a second section having an outer diameter that is not in contact with an inner surface of the needle. The method may also include cutting tissue within the eye with the cutter. The cutter may include a side cutout in the distal end configured to reduce friction between the needle and the cutter. The side cutout may be formed in a lower portion of the distal end. The vitrectomy probe may also include a housing that is attached to proximal ends of the cutter and the needle. The second section may include two or more flat sections on an outer surface of the distal end. The cutter may have a bend ranging from about 2° to about 5° such that the first section of the cutter contacts an inner surface of the needle while the needle is cutting tissue. The method may also include receiving the tissue in a lateral port in the tubular body and receiving additional tissue in a distal port in the distal end. The method may also include aspirating dissected tissue through an interior channel of the cutter. Cutting may include moving the cutter axially back and forth within the needle. The first section may contact the needle while moving, while the second section does not contact the cutter while moving.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

These drawings illustrate certain aspects of some of the embodiments of the present disclosure and should not be used to limit or define the disclosure.

FIGS. 1 and 2 illustrate a vitrectomy probe in accordance with embodiments of the present disclosure.

FIG. 3A illustrates a detailed view of a cutter in accordance with embodiments of the present disclosure.

FIG. 3B illustrates a cutter in accordance with embodiments of the present disclosure.

FIG. 3C illustrates a side cutout on a cutter in accordance with embodiments of the present disclosure.

FIG. 3D illustrates a cutter with an enlarged diameter in accordance with embodiments of the present disclosure.

FIGS. 4A-4D illustrate a cutting cycle in accordance with embodiments of the present disclosure.

FIG. 5 illustrates a vitrectomy probe in accordance with embodiments of the present disclosure.

FIG. 6 illustrates a stroke limiter in accordance with embodiments of the present disclosure.

FIG. 7 illustrates a cutter being inserted into a posterior segment of an eye in accordance with embodiments of the present disclosure.

### DETAILED DESCRIPTION

Embodiments may generally relate to a vitrectomy probe and associated methods of use. More particularly, embodiments may generally relate to vitrectomy probes with a cutter that may have a formed cutting end which may minimize friction with the needle. The vitrectomy probe may or may not have a dual blade. As disclosed herein, embodiments of the vitrectomy probes may comprise cutters that have one or more features configured to reduce friction with the needle while also maintaining sufficient contact with the needle for cutting. For example, by “bulging” or enlarging one or more sections of the outer diameter of the cutter at its distal end to closely match the inner diameter of the needle, full contact along the cutting edges may be achieved which may be needed for proper cutting. By way of further example, the sections of the cutter at its distal end that were not enlarged, may not contact the inner surface of the needle, which may then minimize friction. Furthermore, a side cutout may be added on the lower portion of the sections of the cutter with increased outer diameter, thus reducing the amount of the distal end that may contact the inner surface of the needle, further reducing friction between the needle and the cutter. Cutting edges of the cutter and an inner surface of the needle may make full contact with each other which may benefit a single and a dual blade cutter design. The friction at the distal end may be minimized to only the surfaces that are cutting. In some embodiments, the design of the cutter may be adaptable to a bevel cutter.

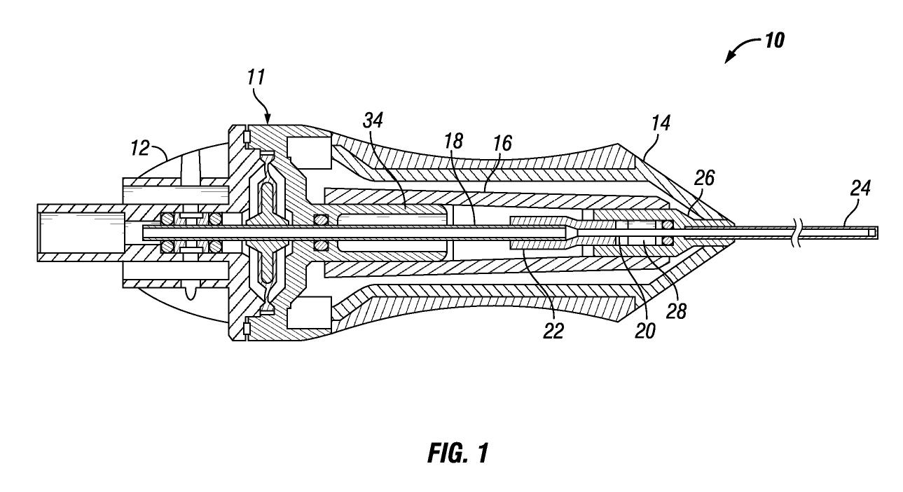

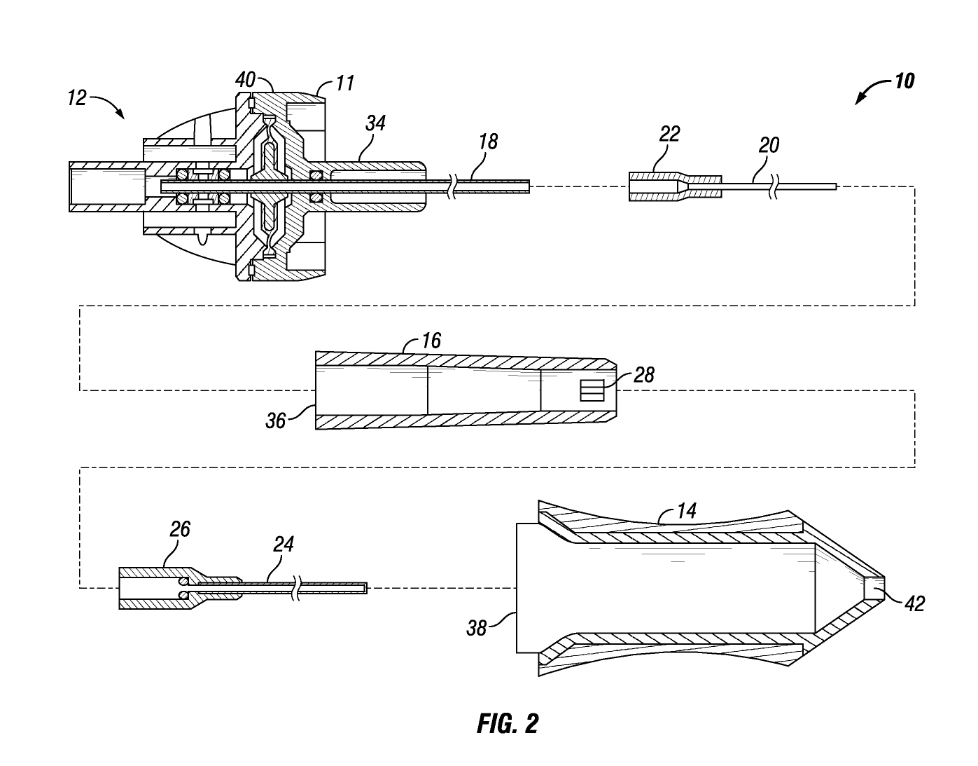

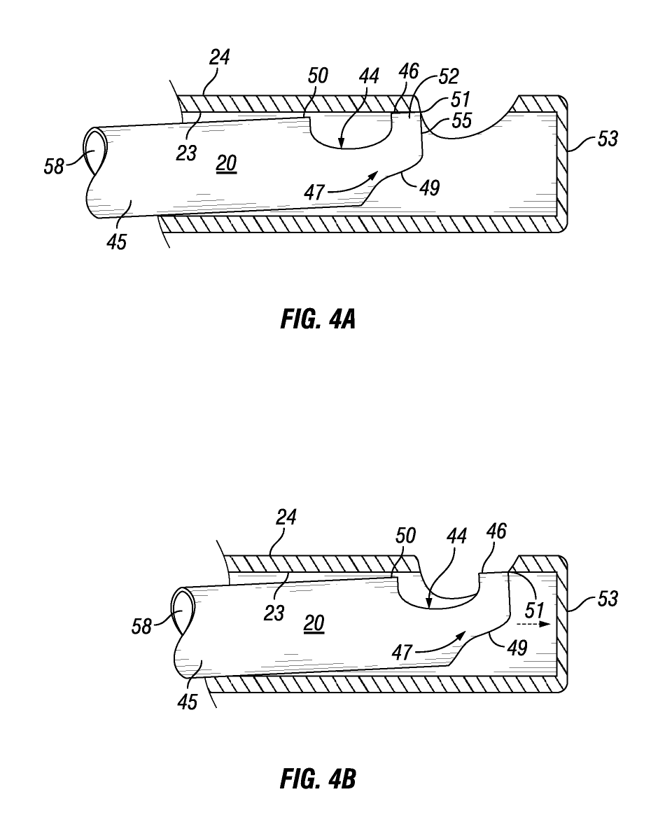

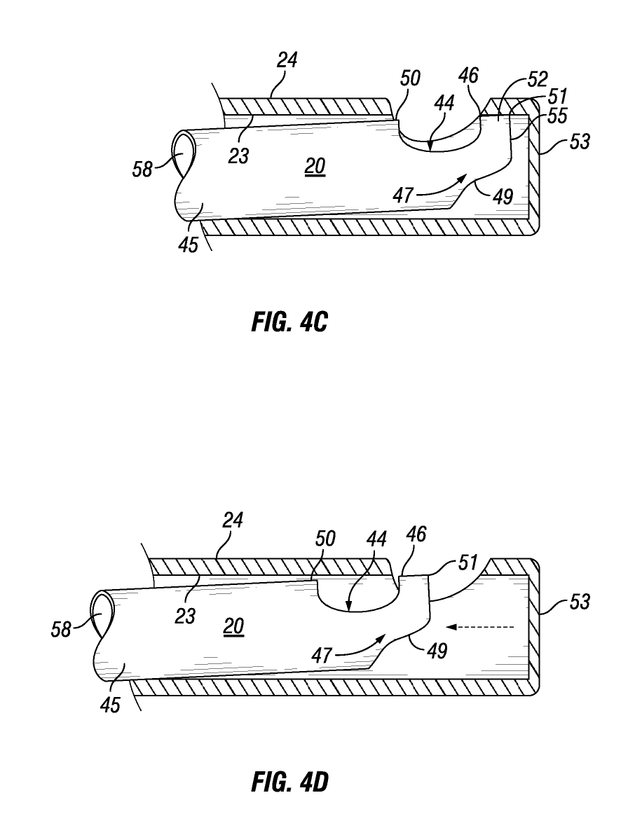

FIGS. 1 and 2 illustrate an example of a vitrectomy probe 10. Vitrectomy probe 10 may comprise housing 11, cutter 20, and needle 24. In the illustrated embodiment, cutter 20 and needle 24 may be attached to housing 11 at their proximal ends. As illustrated, housing 11 may comprise engine 12, shell 14, needle holder 16, and drive shaft 18. As illustrated, cutter 20 may include a cutter coupling 22 at its proximal end. In some embodiments, cutter coupling 22 may be integrally formed on the proximal end of cutter 20. As illustrated, needle 24 may include a needle coupling 26 at its proximal end. In some embodiments, needle coupling 26 may be integrally formed on the proximal end of needle 24. The cutter 20 and needle 24 may both be tubular in shape with a hollow bore. As illustrated, cutter 20 may be slidably disposed in needle 24. Cutter 20 and needle 24 may both extend from housing 11. In operation, cutter 20 may oscillate within needle 24 in response to engine 12. Operation of cutter 20 and needle 24 for cutting tissue will be described in more detail below with respect to FIG. 4.

In the illustrated embodiment, drive shaft 18 may extend axially from engine 12. Distal end of drive shaft 18 may engage cutter coupling 22 of cutter 20. Drive shaft support member 34 may engage opening 36 (best seen on FIG. 2) of needle holder 16. Drive shaft support member 34, drive shaft 18, and cutter coupling 22 may be at least partially disposed within needle holder 16. Bushing 28 within needle holder 16 may engage needle coupling 26 of needle 24, such that cutter 20 may be slidably disposed within needle 24. Needle coupling 26, needle holder 16, cutter coupling 22, drive shaft 18, and drive shaft support member 34 may be disposed within shell 14. As best seen on FIG. 2, shell 14 may contain proximal opening 38 which may engage body 40 of engine 12. Needle 24 may extend through distal opening 42 in shell 14.

Engine 12 may be any type of engine suitable for driving vitrectomy probe 10, such as, for example, a pneumatic engine, a hydraulic engine or an electric engine, among others. Drive shaft 18 may be made of any suitable material, such as, for example, stainless steel. Needle 24 and cutter 20 may be made of any material suitable for posterior segment ophthalmic surgery, such as, for example, surgical stainless steel. Shell 14, needle coupling 26, and cutter coupling 22 may be made from a lightweight material such as, for example, aluminum or rigid plastic. Power may be supplied to the vitrectomy probe 10 via a power cable (not shown). The power cable may be coupled to a surgical console (not shown), and the surgical console may be operable to adjust the power applied to the vitrectomy probe 10 based, for example, on an input to the surgical console by a user, such as, for example, a surgeon. Input from a user to the surgical console may be provided via an input device (not shown), such as, for example, a touch screen, button, slider, footswitch, and/or other input device.

In accordance with embodiments, the modular design of vitrectomy probe 10 may allow a surgeon to quickly and easily switch between building probes of various gauges for needle 24, such as, for example, a vitrectomy probe 10 with needle 24 that is 20 gauge, 23 gauge, 25 gauge, or 27 gauge. More specifically, the ability to quickly attach and remove shell 14 from body 40 of engine 12, to quickly couple different gauges of needle 24 with needle holder 16 and cutter 20 via needle coupling 26 and bushing 28, and to quickly couple cutter 20 to drive shaft 18 via cutter coupling 22 may greatly increase manufacturing flexibility and simplify the assembly process of vitrectomy probe 10. It should be understood that FIGS. 1 and 2 illustrate an example configuration of vitrectomy probe 10 and that other suitable configurations may be suitable for use with the design features of cutter 20 disclosed herein.

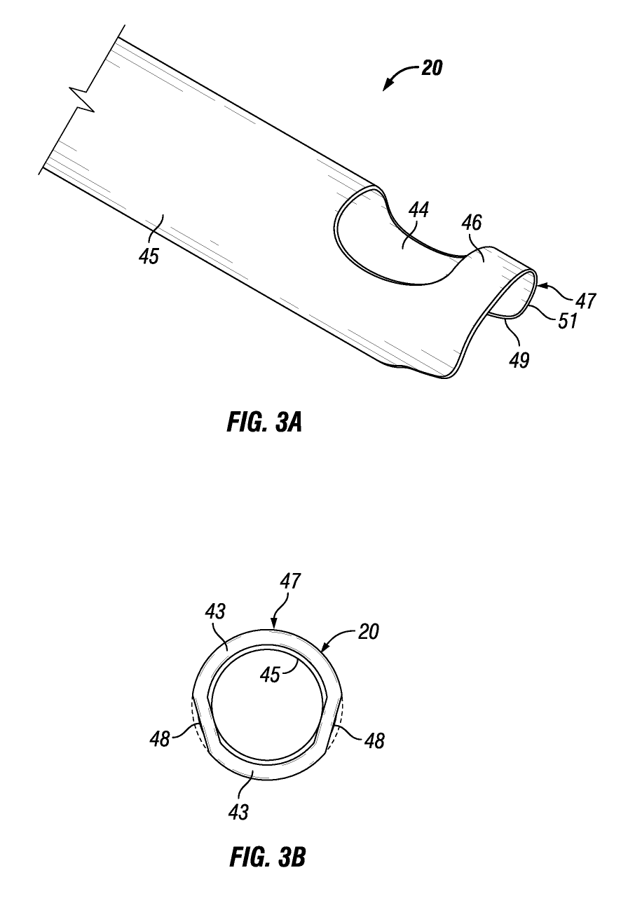

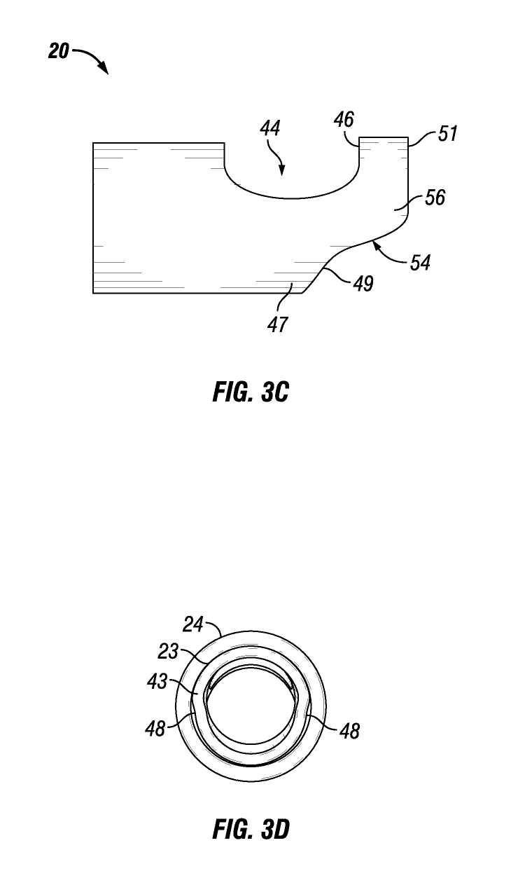

FIG. 3A illustrates a detailed view of cutter 20 in accordance with embodiments of the present disclosure. Cutter 20 may include a tubular body 45 having a distal end 47. Without limitation, tubular body 45 may be in the form of a hollow tube (e.g., a hollow cylinder), but other configurations of tubular body 45 may also be suitable. As illustrated, a lateral port 44 may be formed in tubular body 45 that may receive various materials, such as tissue, during operation. In some embodiments, the tissue may be ophthalmic tissue, such as vitreous and/or membrane. In the illustrated embodiment, lateral port 44 may be formed in lateral sides of tubular body 45. Lateral port 44 may be of a polygonal (e.g., rectangular) or other suitable shape. As illustrated, tubular body 45 may be open at distal end 47, wherein distal end 47 includes distal cutting edge 51 to cut tissue, and distal port 49 to receive cut tissue, such as vitreous and/or membrane.

Cutter 20 may in the form of a single blade configuration or a dual blade configuration including two cutting edges, proximal cutting edge 46 and distal cutting edge 51. Proximal cutting edge 46 may be formed by a distal side of lateral port 44. Distal cutting edge 51 may be formed by a distal side of distal end 47. When moving, the proximal cutting edge 46 and distal cutting edge 51 may cut material, such as tissue. For example, the proximal cutting edge 46 and distal cutting edge 51 may cooperate with cutting edges on needle 24 to cut the material.

Without limitation, cutter 20 may include a guillotine blade, a hole blade, or a slit blade. A guillotine blade may include a blade resembling a guillotine that covers lateral port 44 entirely when closed. A hole blade may include a hole with a diameter, for example, ranging from about 0.1 mm (millimeters) to about 0.3 mm, that corresponds to the center of lateral port 44 when the blade closes. A slit blade may include a slit with a width, ranging from about 0.1 mm to about 0.3 mm wide, for example, that corresponds to the center of lateral port 44 when the blade closes.

Cutter 20 may be made of any suitable material, including surgical stainless steel. Tubular body 45 of cutter 20 may have any suitable dimensions, including without limitation a length of about 1 inch to about 2 inches (e.g., 1.7 inches) and an outer diameter of about 0.004 inches to about 0.025 inches (e.g., an outer diameter of 0.0119 inches, 0.0146 inches, or 0.0190 inches). Tubular body 45 may include a bend of about 2° to about 5°. Without limitation, the bend may be configured to contact at least a portion of the needle 24 and cutter 20. In some embodiments, the bend length may be about 0.01 inch to about 0.2 inch. Additionally, in some embodiments, tubular body 45 of cutter 20 may have a size that ranges from about 23 gauge to about 27 gauge. Other dimensions are also contemplated.

As previously mentioned, embodiments of cutter 20 may be formed with one or more features that may minimize friction, which will now be described in more detail with respect to FIGS. 3B-3D. FIG. 3B illustrates a cross-sectional end view of cutter 20 taken through distal end 47. Distal end 47 may include a first section 43 and a second section 48. First section 43 may have an outer diameter that is greater than an outer diameter of tubular body 45. As illustrated, first section 43 may include two or more sections. Second section 48 may have an outer diameter such that an outer surface of the second section 48 does not contact inner surface 23 of needle 24. For example, second section 48 may have an outer diameter that is less than an outer diameter of first section and, alternatively, that is less than an outer diameter of tubular body 45. As illustrated, second section 48 may include two or more sections which may be flat, for example. Because first section 43 may have an outer diameter that is greater than an outer diameter of tubular body 45, full contact along proximal cutting edge 46 and distal cutting edge 51 may be achieved, which may be needed for proper cutting. Because second section 48 may have an outer diameter that is less than an outer diameter of first section 43, second section 48 may not contact the inner surface of the needle 24 (e.g., shown on FIG. 1), which may then minimize friction. The outer diameter of cutter 20 may be enlarged to closely match the inside diameter of needle 24, while two or more sides of cutter 20 may be flattened (e.g., second side 48) to remove contact with needle inner surface 23. The second section 48 may not make contact with needle inner surface 23, and therefore, remove friction.

Cutter 20 may also include a side cutout 54 on a lower distal portion 56 of cutter 20. The side cutout 54 may be formed by cutting distal end 47 (e.g., electrical discharge machining) or the distal end 47 may be formed to include side cutout 54. The side cutout 54 may enlarge distal port 49 and may extend longitudinally along the z-axis of distal end 47. The side cutout 54 may contribute to friction removal as the cutter 20 axially moves back and forth (i.e., cutting) within needle 24. By way of example, by removing material from cutter 20, and thus, reducing the size of the first section 43 (shown on FIG. 3D) of cutter 20, the contact area between cutter 20 and needle 24 may be reduced. Side cutout 54 may be added on the lower distal portion 56 of the first section 43, since this lower distal portion 56 may contact the needle inner surface 23 (shown on FIG. 3D) and may increase friction. As mentioned above, the cutter 20 may be slidably positioned/disposed inside needle 24.

By “bulging” or enlarging some sections of the diameter (e.g., first section 43) of the cutter 20 to closely match the inner diameter of needle 24 (shown on FIG. 3D), the full contact along the proximal cutting edge 46 and distal cutting edge 51 may be achieved which may be needed for proper cutting. Additionally, sections of the cutter 20 diameter that were not enlarged (e.g., second section 48), may not contact the needle inner surface 23 which may then minimize friction. Proximal cutting edge 46/distal cutting edge 51 and needle inner surface 23 may make full contact with each other, which may benefit a single and a dual blade cutter design. The friction at cutter 20 may be minimized at the surfaces that are cutting (e.g., proximal cutting edge 46 and distal cutting edge 51). In certain embodiments, the design of the cutter 20 may be adaptable to a bevel cutter.

FIG. 4A illustrates cutter 20 positioned within needle 24. Cutter 20 may contact the needle inner surface 23 at two or more portions of contact (e.g., contact portions 50, 52, 55). Contact portion 50 may be located at a portion of tubular body 45 adjacent to lateral port 44, contact portion 52 may be located at the proximal cutting edge 46, and contact portion 55 may be located at distal cutting edge 51. As cutter 20 moves axially back and forth (i.e., cutting motion) within needle 24, only contact portions 50, 52, 55 may contact needle inner surface 23, thereby reducing friction and allowing cutter 20 to cut more efficiently (e.g., less buildup of heat, less resistance to axial movement of cutter 20). In operation, tissue may enter into the cutter 20 through the lateral port 44 and be dissected by the proximal cutting edge 46 and distal cutting edge 51 as the cutter 20 moves axially back and forth to cut tissue. A vacuum may also be generated within an interior channel 58 (a vacuum source, not shown, may be fluidly connected to interior channel 58 to provide aspiration) of the cutter 20 to aspirate the dissected tissue.

FIGS. 4A-4D illustrate a cutting cycle with the cutter 20. FIG. 4A represents a stage in the cutting cycle where cutter 20 is in the open position. In this open position, vacuum pressure in interior channel 58 may pull tissue into distal port 49. As shown in FIG. 4B, cutter 20 may travel distally towards distal end 53. As cutter 20 moves forward, distal cutting edge 51 may cut tissue that has entered distal port 49. The severed tissue may be pulled through interior channel 58 by an aspiration system. As illustrated in FIG. 4C, the cutter 20 may continue to move distally further into needle 24. While not shown, cutter may move until distal cutting edge 51 becomes substantially flush with distal end 53 of needle 24. After cutter 20 moves distally towards distal end 53, cutter 20 may move proximally (backwards, i.e., away from distal end 53), as illustrated in FIG. 4D. As cutter 20 moves proximally, proximal cutting edge 46 may cut tissue that has entered lateral port 44. The severed tissue may be pulled through interior channel 58 by an aspiration system.

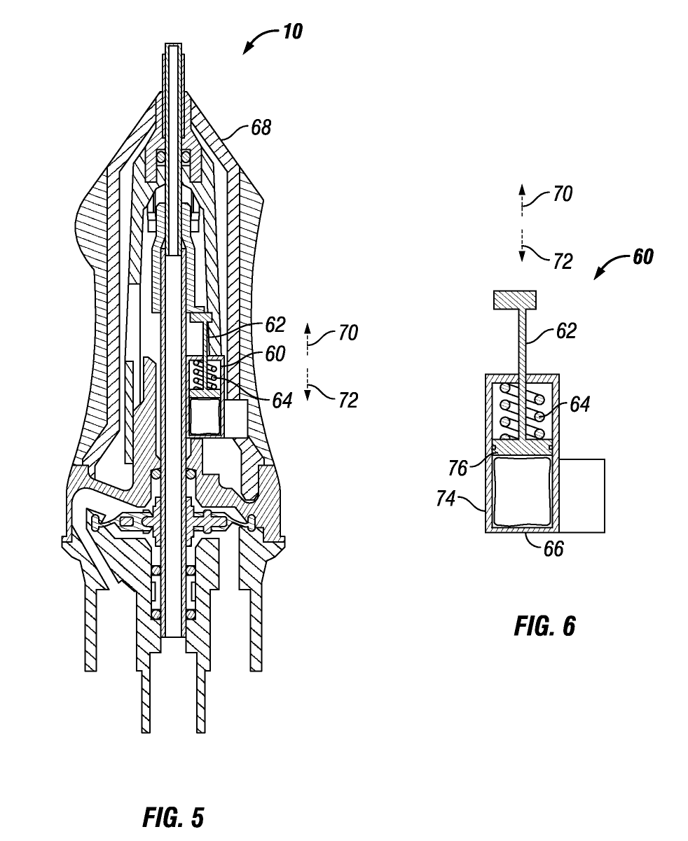

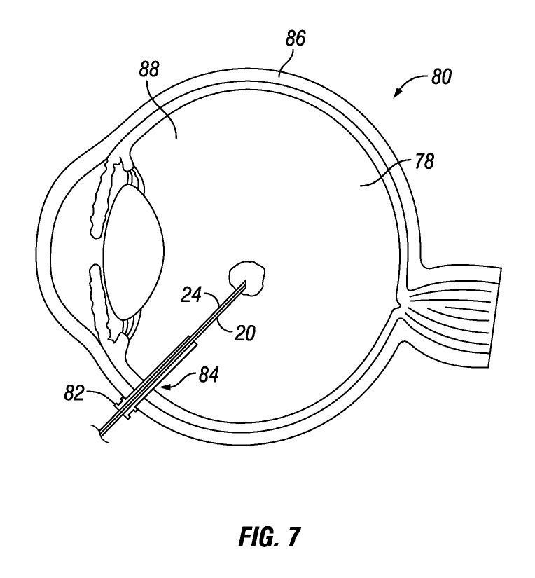

Referring now to FIGS. 5 and 6, embodiments of vitrectomy probe 10 may include a stroke limiter 60 in order to limit the range of motion or “stroking” of cutter 20 in order to prevent damage to the needle 24 and cutter 20. Stroke limiter 60 may include a push rod 62, a spring 64, and an enclosure 66. In some embodiments, the enclosure 66 may be fixed relative to the housing 68. The push rod 62 may be moveable relative to the enclosure 66. Further, in some embodiments, the spring 64 may be omitted.

Movement of the push rod 62 in a direction indicated by arrow 70 may be accomplished, for example, by applying a voltage to a peltier cooler to heat fluid contained in portion 74. The expanding fluid applies pressure to the piston 76 and, therefore, a force on the piston 76 urging the push rod 62 to move in a direction of arrow 70. In embodiments including the spring 64, the spring 64 may apply an opposing force in the direction of arrow 72. The push rod 62 may move in the direction of arrow 70 when the force exerted on the push rod 62 by the fluid exceeds the biasing force of the spring 64. In embodiments containing no spring 64, the push rod 62 may move without influence of a spring force.

Additionally, the push rod 62 may be moved in the direction of arrow 72 by decreasing or removing the voltage from a peltier cooler and allowing the fluid within portion 74 to cool or by applying a voltage opposite the voltage to move the push rod 62 in the direction of arrow 72. As the fluid cools, the fluid contracts, reducing the force applied to the push rod 62, and, therefore, causing the push rod 62 to move in the direction of arrow 72. Where a spring 64 may be present, the force applied by the spring 64 may urge the push rod 62 in the direction of arrow 72.

Referring now to FIG. 7, use of cutter 20 in an ophthalmic surgical procedure will now be described in accordance with embodiments of the present disclosure. As illustrated in FIG. 7, during an ophthalmic surgical procedure, such as a retinal surgical procedure, the cutter 20 and needle 24 may be inserted into the posterior segment 78 of the eye 80, such as through a cannula 82 disposed in an incision 84 through the sclera 86 of the eye 80, to remove and aspirate ophthalmic tissue, such as vitreous and/or membrane. For example, during a retinal surgical procedure, the needle 24 with cutter 20 disposed therein cutter 20 may be inserted into the posterior segment 78 of the eye 80. Cutter 20 may be operated to remove the ophthalmic tissue, which may include be vitreous humor 88 (interchangeably referred to as “vitreous”) , a jelly-like substance that occupies the volume defined by the posterior segment 78, as cutter 20 moves back and forth within needle 24. The cutter 20 may also be used to remove membranes covering the retina or other tissues. Dissected tissue may be removed via interior channel 58, as mentioned above (e.g., shown in FIGS. 4A-4D).

It is believed that the operation and construction of the present disclosure will be apparent from the foregoing description. While the apparatus and methods shown or described above have been characterized as being preferred, various changes and modifications may be made therein without departing from the spirit and scope of the disclosure as defined in the following claims.

What is claimed is:

1. A vitrectomy probe comprising:
a needle; and
a cutter slidably disposed within the needle, wherein the cutter comprises a tubular body having a distal end, wherein the distal end comprises a first section having an outer diameter that is greater than an outer diameter of the tubular body and a second section having an outer surface that is not in contact with an inner surface of the needle.

2. The vitrectomy probe of claim 1, wherein the cutter comprises a side cutout in the distal end, wherein the side cutout is configured to reduce friction between the needle and the cutter.

3. The vitrectomy probe of claim 2, wherein the side cutout is formed in a lower portion of the distal end.

4. The vitrectomy probe of claim 1, wherein the cutter comprises a lateral port in the tubular body.

5. The vitrectomy probe of claim 4, wherein the cutter comprises a distal port in the distal end of the tubular body.

6. The vitrectomy probe of claim 5, wherein the cutter further comprises a proximal cutting edge and a distal cutting edge, wherein the proximal cutting edge is formed by a distal side of the lateral port, and wherein the distal cutting edge is formed by a distal side of the distal end.

7. The vitrectomy probe of claim 1, wherein the second section comprises two or more flat sections on an outer surface of the distal end.

8. The vitrectomy probe of claim 1, wherein the cutter has a bend ranging from about 2° to about 5°.

9. The vitrectomy probe of claim 8, wherein the bend is configured to allow a contact portion between the inner surface of the needle and the first section of the cutter.

10. The vitrectomy probe of claim 1, further comprising a housing, wherein the housing is attached to proximal ends of the cutter and the needle.

11. The vitrectomy probe of claim 1, further comprising an interior channel of the cutter to aspirate dissected tissue.

12. A method for operating a vitrectomy probe, the method comprising:
positioning the vitrectomy probe in an eye, wherein the vitrectomy probe comprises:
a needle; and
a cutter slidably disposed within the needle, wherein the cutter comprises a tubular body and a distal end, wherein the distal end comprises a first section having an outer diameter that is greater than an outer diameter of the tubular body and a second section having an outer surface that is not in contact with an inner surface of the needle; and
cutting tissue within the eye with the cutter.

13. The method of claim 12, wherein the cutter comprises a side cutout in the distal end configured to reduce friction between the needle and the cutter.

14. The method of claim 13, wherein the side cutout is formed in a lower portion of the distal end.

15. The method of claim 12, wherein the vitrectomy probe further comprises a housing, wherein the housing is attached to proximal ends of the cutter and the needle.

16. The method of claim 12, wherein the second section comprises two or more flat sections on an outer surface of the distal end.

17. The method of claim 12, wherein the cutter has a bend ranging from about 2° to about 5° such that the first section of the cutter contacts the inner surface of the needle during cutting of tissue.

18. The method of claim 12, further comprising receiving the tissue in a lateral port in the tubular body and receiving additional tissue in a distal port in the distal end.

19. The method of claim 12, further comprising aspirating dissected tissue through an interior channel of the cutter.

20. The method of claim 12, wherein cutting comprises moving the cutter axially back and forth within the needle, wherein the first section contacts the needle during movement, while the second section does not contact the cutter during movement.
